(19) United States
(12) Patent Application Publication
(10) Pub. No.: US 2009/0091244 A1
(43) Pub. Date: Apr. 9, 2009

(54) AMINE COMPOUND, ORGANIC LIGHT-EMITTING DEVICE, AND ORGANIC BLUE-LIGHT-EMITTING DEVICE

(75) Inventors: Chika Negishi, Yokosuka-shi (JP); Naoki Yamada, Inagi-shi (JP); Keiji Okinaka, Kawasaki-shi (JP); Minako Nakasu, Tokyo (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/912,621

(22) PCT Filed: Dec. 13, 2006
(86) PCT No.: PCT/JP2006/325318

§ 371 Date: Oct. 25, 2007

(30) Foreign Application Priority Data
Dec. 20, 2005 (JP) 2005-366557
Nov. 20, 2006 (JP) 2006-312927

(51) Int. Cl. H01J 1/63; C07C 211/57; C07D 333/50

(52) U.S. Cl. 313/504; 564/308; 549/43

(57) ABSTRACT

To realize extremely high luminous efficiency and to provide an organic light-emitting device having a optical output with a long lifetime. Provided is an amine compound represented by the general formula (1).

### TECHNICAL FIELD

The present invention relates to an organic light-emitting device, and more specifically, to a device that radiates light by applying an electric field to a thin film composed of an organic compound.

### BACKGROUND ART

An organic light-emitting device is a device including a thin film which contains a fluorescent organic compound or a phosphorescent organic compound and which is interposed between an anode and a cathode, wherein a hole and an electron are injected from respective electrodes.

Further, an exciton of the fluorescent compound or the phosphorescent compound is generated in the device, and the device uses light radiated when the exciton returns to its ground state.

The recent progress of an organic light-emitting device is significant, and it is suggested that the device can be applied to a wide variety of applications because of the following reasons. The device shows a high luminance at a low applied voltage. In addition, the device has a variety of emission wavelengths. Furthermore, the device can be a thin, light-weight light-emitting device with high-speed responsiveness.

However, at present, an optical output with additionally high luminance, or additionally high conversion efficiency has been needed.

Japanese Patent No. 2851185 discloses, as examples of a material for an organic light-emitting device such as an amino compound and an organic light-emitting device using the material, an organic light-emitting device using a diamino condensed aromatic ring derivative. In addition, Japanese Patent No. 3508984 and Japanese Patent Application Laid-Open No. 2000-016973 disclose, as other examples, organic light-emitting devices using a diaminofluorene derivative. In addition, Japanese Patent Application Laid-Open No. H10-251633 and Japanese Patent Application Laid-Open No. 2001-39933 disclose, as other examples, organic light-emitting devices using a diamino compound. Further, Japanese Patent Application Laid-Open No. 2005-320277 discloses, as another example, an organic light-emitting device using a diamino compound as a quarter phenylene derivative in which a central biphenylene group is of an ortho-substituted type.

### DISCLOSURE OF THE INVENTION

The present invention has been made in order to solve the above-mentioned problems of the prior art, and an object of the present invention is to realize extremely high luminous efficiency and to provide an organic light-emitting device having an optical output with a long lifetime. Another object of the present invention is to provide an organic light-emitting device that can be easily produced at a relatively low cost.

The inventors of the present invention have made extensive studies in order to solve the above-mentioned problems. As a result, they have accomplished the present invention.

(1) According to the present invention, there is provided an amine compound represented by the following general formula (1):

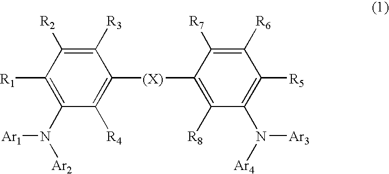

wherein X represents the following general formula (2):

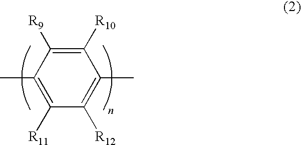

Ar₁, Ar₂, Ar₃ and Ar₄ each independently represent a substituted or unsubstituted aryl group, or a substituted or unsubstituted heterocyclic group, and may be the same or different, and at least one of Ar₁, Ar₂, Ar₃ and Ar₄ represents a substituted or unsubstituted fluorenyl group;

R₁ to R₁₂ each represent a hydrogen atom, a halogen atom, a substituted or unsubstituted alkyl group, a substituted or unsubstituted alkoxy group, a substituted or unsubstituted aryl group, or a substituted or unsubstituted heterocyclic group, and may be the same or different;

n represents an integer of 0 to 10; and when a plurality of R₉'s, a plurality of R₁₀'s, a plurality of R₁₁'s, or a plurality of R₁₂'s, are present, the plurality of R₉'s, the plurality of R₁₀'s, the plurality of R₁₁'s, or the plurality of R₁₂'s may be the same or different, or two of R₉'s to R₁₂'s may form a ring, or X represents a single bond when n represents 0.

(2) According to the present invention, there is also provided an amine compound according to the above item (1), wherein X in the general formula (1) is represented by the following general formula (3):

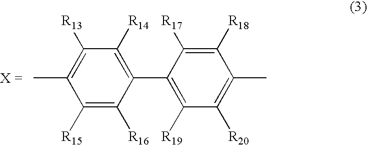

wherein R₁₃ to R₂₀ each represent a hydrogen atom, a halogen atom, a substituted or unsubstituted alkyl group, a substituted or unsubstituted alkoxy group, a substituted or unsubstituted aryl group, or a substituted or unsubstituted heterocyclic group, and may be the same different.

(3) According to the present invention, there is also provided an amine compound according to the above item (1), wherein X in the general formula (1) is represented by the following general formula (4):

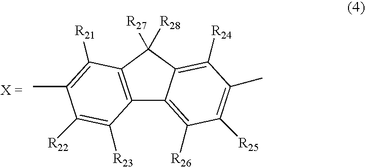

wherein R₂₁ to R₂₈ each represent a hydrogen atom, a halogen atom, a substituted or unsubstituted alkyl group, a substituted or unsubstituted alkoxy group, a substituted or unsubstituted aryl group, or a substituted or unsubstituted heterocyclic group, and may be the same or different.

(4) According to the present invention, there is also provided an amine compound according to the above item (1), wherein X in the general formula (1) is represented by the following general formula (5):

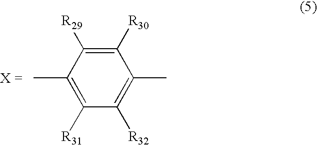

wherein R₂₉ to R₃₂ each represent a hydrogen atom, a halogen atom, a substituted or unsubstituted alkyl group, a substituted or unsubstituted alkoxy group, a substituted or unsubstituted aryl group, or a substituted or unsubstituted heterocyclic group, and may be the same or different.

(5) According to the present invention, there is provided an amine compound according to Item (1), in which X in the general formula (1) represents a single bond.

(6) According to the present invention, there is provided an organic light-emitting device, including an organic compound layer interposed between a pair of electrodes composed of an anode and a cathode, at least one of the anode and the cathode being transparent or semi-transparent, wherein the organic compound layer contains an amine compound according to the above item (1).

(7) According to the present invention, there is provided an organic light-emitting device, including an organic compound layer as a hole transport layer or a hole injection layer interposed between a pair of electrodes composed of an anode and a cathode, at least one of the anode and the cathode being transparent or semi-transparent, wherein the hole transport layer or the hole injection layer contains an compound according to the above item (1).

(8) According to the present invention, there is provided an organic light-emitting device, including an organic compound layer as a light-emitting layer interposed between a pair of electrodes composed of an anode and a cathode at least one of the anode and the cathode being transparent or semi-transparent, wherein the light-emitting layer contains an amino compound according to Item (1).

(9) According to the present invention, there is provided an organic blue-light-emitting device, including an organic compound layer interposed between a pair of electrodes composed of an anode and a cathode, at least one of the anode and the cathode being transparent or semi-transparent, wherein a relationship of (an absolute value of an ionization potential of a hole transport material)−(an absolute value of an electron affinity of the hole transport material)≧3.20 eV is established; and a relationship of (an absolute value of an electron affinity of a host material for use in a light-emitting layer)−(the absolute value of the electron affinity of the hole transport material)≧0.42 eV is established.

(10) According to the present invention, there is provided an organic blue-light-emitting device according to the above item (9), wherein a relationship of ((the absolute value of the ionization potential of the hole transport material)−(the absolute value of the electron affinity of the hole transport material))−((an absolute value of an ionization potential of the host material for use in the light-emitting layer)−(the absolute value of the electron affinity of the host material for use in the light-emitting layer))≧0.25 eV is established.

(11) According to the present invention, there is provided a blue organic light-emitting device according to Item (9), wherein the organic compound for use in a hole transport layer is a triarylamine derivative.

(12) According to the present invention, there is provided an organic blue-light-emitting device, including an organic compound layer interposed between a pair of electrodes composed of an anode and a cathode, at least one of the anode and the cathode being transparent or semi-transparent, wherein an organic compound used for a hole transport layer contains at least one kind of an amine compound according to the above item (1).

The amine compound represented by the general formula (1) of the present invention emits light with a high efficiency at a low applied voltage. In addition, the device provides excellent durability.

### BRIEF DESCRIPTION OF THE DRAWINGS

FIG. 1 is a sectional view showing an example of an organic light-emitting device of the present invention;

FIG. 2 is a sectional view showing another example of the organic light-emitting device of the present invention;

FIG. 3 is a sectional view showing still another example of the organic light-emitting device of the present invention; and

FIG. 4 is a sectional view showing a further example of the organic light-emitting device of the present invention.

FIG. 5 is a sectional view showing a still further example of the organic light-emitting device of the present invention.

### BEST MODE FOR CARRYING OUT THE INVENTION

A compound represented by the above-described general formula (1) can be used mainly as a material for a hole transport layer or a material for a hole injection layer. The compound is characterized in that, when the compound is used as a material for a hole transport layer among such materials, the compound can be used to serve as an electron blocking layer, and the compound for use in a light-emitting device can provide the device having a high luminous efficiency and a long lifetime and have an effect particularly on highly efficient light emission.

A molecule of the compound represented by the general formula (1) was designed for the purpose of improving the electron blocking property of a hole transport layer. In other words, the molecule was designed while the fact that the absolute value of the electron affinity of the hole transport layer should be sufficiently smaller than the absolute value of the electron affinity of a light-emitting layer was taken into consideration.

Details about the foregoing are as described below. Molecular design using a meta-phenylene group was performed for breaking a n-conjugated bond in a phenylene group on an amino substituent in a molecule of the compound. As a result, a material for a hole transport layer having a wide band gap ((the absolute value of the ionization potential of a hole transport layer)−(the absolute value of the electron affinity of the hole transport layer)) (hereinafter referred to as “Y”) and a small absolute value of an electron affinity, and a hole transport layer using the material can be formed. In addition, in order that light emission with additionally high efficiency may be expected, the band gap can be adjusted by introducing the substituents R₁ to R₃₂ onto the phenylene group on the amino substituent. Further, the band gap, the ionization potential, and the electron affinity can be adjusted by transforming the substituents Ar₁ to Ar₄ on the respective amino groups.

Detailed, extensive investigation conducted by the inventors of the present invention has found that luminous efficiency tends to be larger as ((the absolute value of the electron affinity of a host material for use in a light-emitting layer)−(the absolute value of the electron affinity of a material for a hole transport layer)) (hereinafter referred to as “Z”) becomes larger. It has also been found that, when Z is 0.42 eV or more and Y is 3.20 eV or more, an effect of improving the luminous efficiency is particularly significant. This is because the leak of an exciton from the light-emitting layer to the hole transport layer can be suppressed better as Y becomes larger. A compound described in the present invention can maintain Y of 3.20 eV or more because the compound uses a meta-phenylene group for cutting a n-conjugated bond.

In addition, it has been found that the increase of the value of Y is effective for an improvement in electron blocking property of the hole transport layer, that is, the increase of the value of Z. Further, it has been found that an improvement in electron blocking property of the hole transport layer requires the widening of a difference in band gap between the hole transport layer and a host of the light-emitting layer. In addition, detailed device evaluation has found that the achievement of highly efficient light emission and a long lifetime requires Y of 3.20 eV or more, Z of 0.42 eV or more, and a difference in band gap between the hole transport layer and the host of the light-emitting layer of 0.25 eV or more. Further, it has been found that a difference in band gap between the hole transport layer and the host of the light-emitting layer of 0.27 eV or more and Z of 0.48 eV or more are more preferable in order that those effects may be exerted. In addition, it has been found that such effects are particularly effective for a blue organic light-emitting device having a wide energy gap among an organic red-light-emitting device, an organic green-light-emitting device, and the organic blue-light-emitting device. As shown in examples and comparative examples, the above numerical values for Y, Z and the like were derived as a result of detailed investigation and discussion.

In addition, when a fluorescent material or a phosphorescent material is used in a light-emitting layer, each of a blue-light-emitting layer, a green-light-emitting layer, and a red-light-emitting layer can be transformed into a hole transport layer having an appropriate ionization potential and an appropriate electron affinity. In addition, when a fluorene derivative group is introduced into any one of Ar₁ to Ar₄, carrier transport property can be improved, Tg can be increased, and a material having good thermostability can be obtained.

In addition, when a phosphorescent material is used in a light-emitting layer, a hole transport layer in contact with the light-emitting layer desirably has an energy higher than that of the T1 level of the lowest excited triplet state of the phosphorescent material. To this end, a biphenylene group, fluorenyl group or phenylene group having a high T1 energy level, or a single bond is preferably introduced into X. More preferable examples include a phenylene group and a single bond. In addition, in consideration of the injection property of a hole from an anode, a biphenylene group or phenylene group represented by X is preferably para-substituted because the absolute value of the ionization potential of the hole transport layer must be small.

In addition, the compound represented by the general formula (1) can be used also as a material for a blue-light-emitting layer having high color purity. The present invention has been made by molecular design based on such discussion as described above.

Hereinafter, the present invention will be described in detail.

Specific examples of the substituents in the general formula (1) will be shown below.

According to the general formula (1), examples of the substituted or unsubstituted alkyl group include, of course not limited to, a methyl group, an ethyl group, a n-propyl group, an isopropyl group, an n-butyl group, a tert-butyl group, a sec-butyl group, an octyl group, a 1-adamantyl group, and a 2-adamantyl group.

Examples of the substituted or unsubstituted aryl group include, of course not limited to, a phenyl group, a naphthyl group, a pentalenyl group, an indenyl group, an azulenyl group, an anthryl group, a pyrenyl group, an indacenyl group, an acenaphthenyl group, a phenanthryl group, a phenalenyl group, a fluoranthenyl group, an acephenanthyl group, an aceanthryl group, a triphenylenyl group, a chrysenyl group, a naphthacenyl group, a perylenyl group, a pentacenyl group, a biphenyl group, a terphenyl group, and a fluorenyl group.

Examples of the substituted or unsubstituted heterocyclic group include, of course not limited to, a thienyl group, a pyrrolyl group, a pyridyl group, an oxazolyl group, an oxadiazolyl group, a thiazolyl group, a thiadiazolyl group, a terthienyl group, a carbazolyl group, an acridinyl group, and a phenanthrolyl group.

Examples of the halogen atom include fluorine, chlorine, bromine, and iodine.

Examples of the substituted or unsubstituted amino group include, of course not limited to, an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tertiary butylphenyl)amino group, and an N-phenyl-N-(4-trifluoromethylphenyl)amino group.

Examples of the substituted or unsubstituted alkoxy group include, of course not limited to, a substituted or unsubstituted methoxy group, ethoxy group, propoxy group, 2-ethyloctyloxy group, phenoxy group, 4-tertiary butylphenoxy group, benzyloxy group, and thienyloxy group.

Examples of substituents which the above-mentioned substituents may have include: alkyl groups such as a methyl group, an ethyl group, and a propyl group; aralkyl groups such as a benzyl group and a phenethyl group; aryl groups such as a phenyl group and a biphenyl group; heterocyclic groups such as a thienyl group, a pyrrolyl group, and a pyridyl group; amino groups such as an N,N-dimethylamino group, an N,N-diethylamino group, an N,N-dibenzylamino group, an N,N-diphenylamino group, an N,N-ditolylamino group, and an N,N-dianisolylamino group; alkoxyl groups such as a methoxyl group, an ethoxyl group, a propoxyl group, and a phenoxyl group; a cyano group; and halogen atoms such as fluorine, chlorine, bromine, and iodine.

Next, exemplified compounds are shown below as representative examples of the compound represented by the general formula (1); provided that the compound is not limited to these compounds.

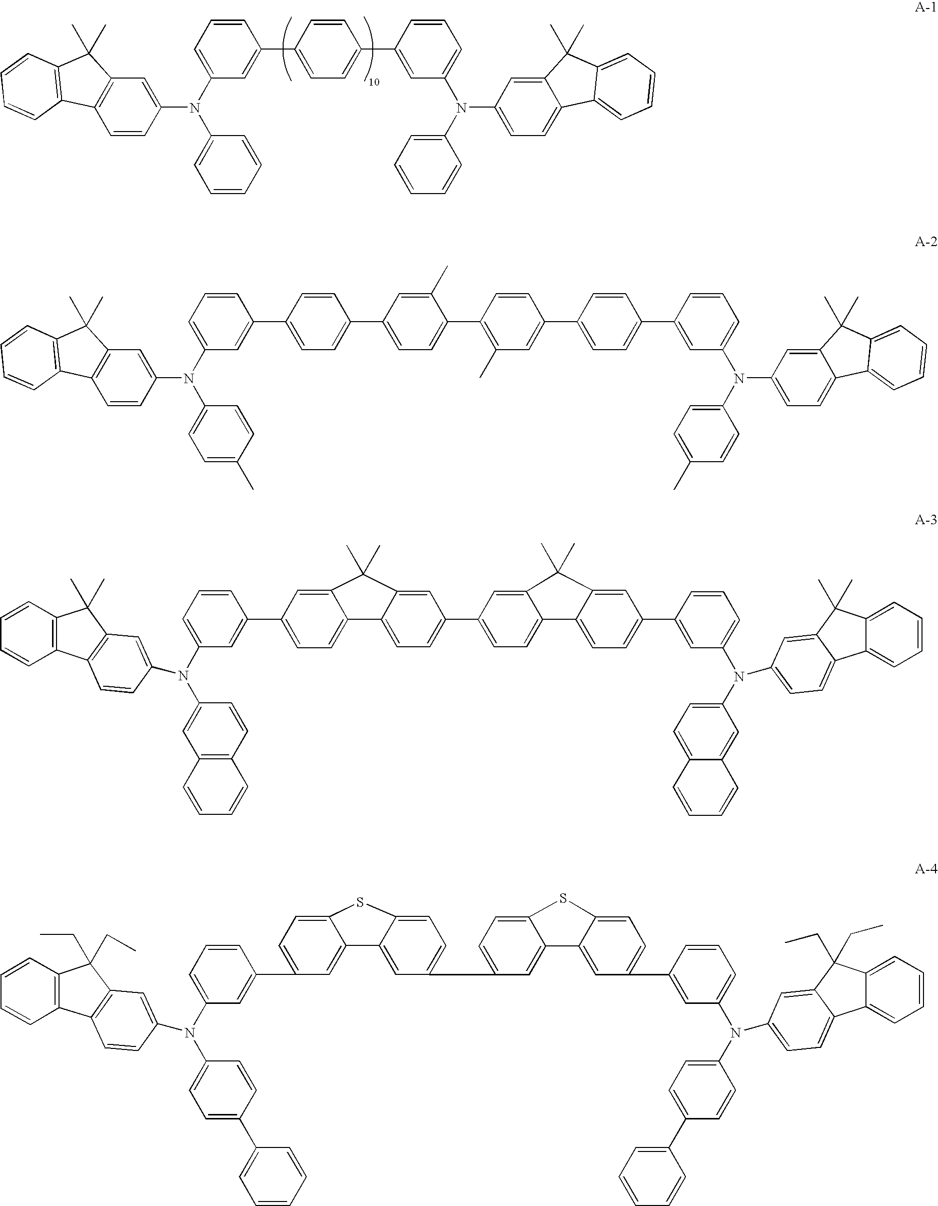

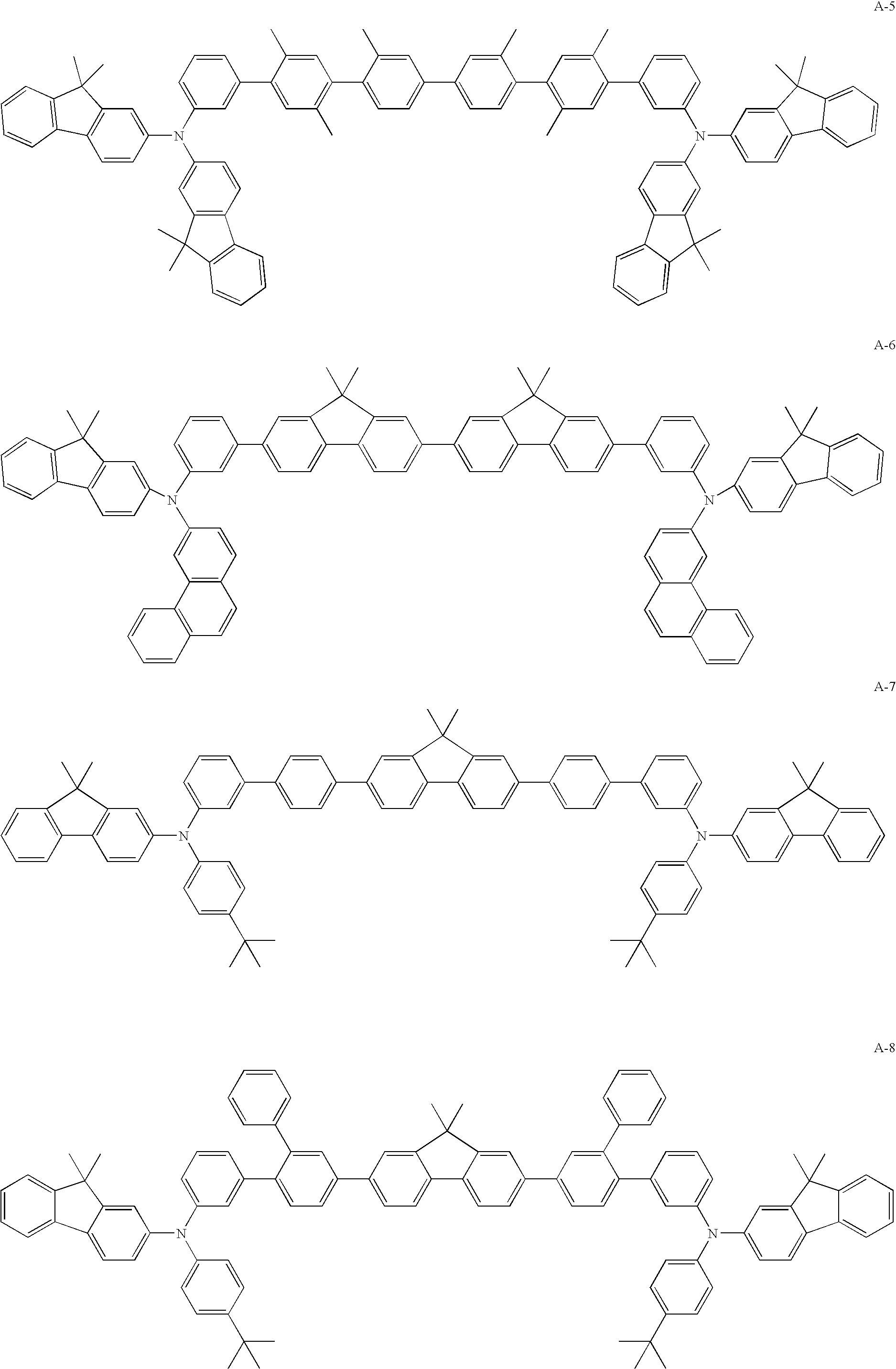

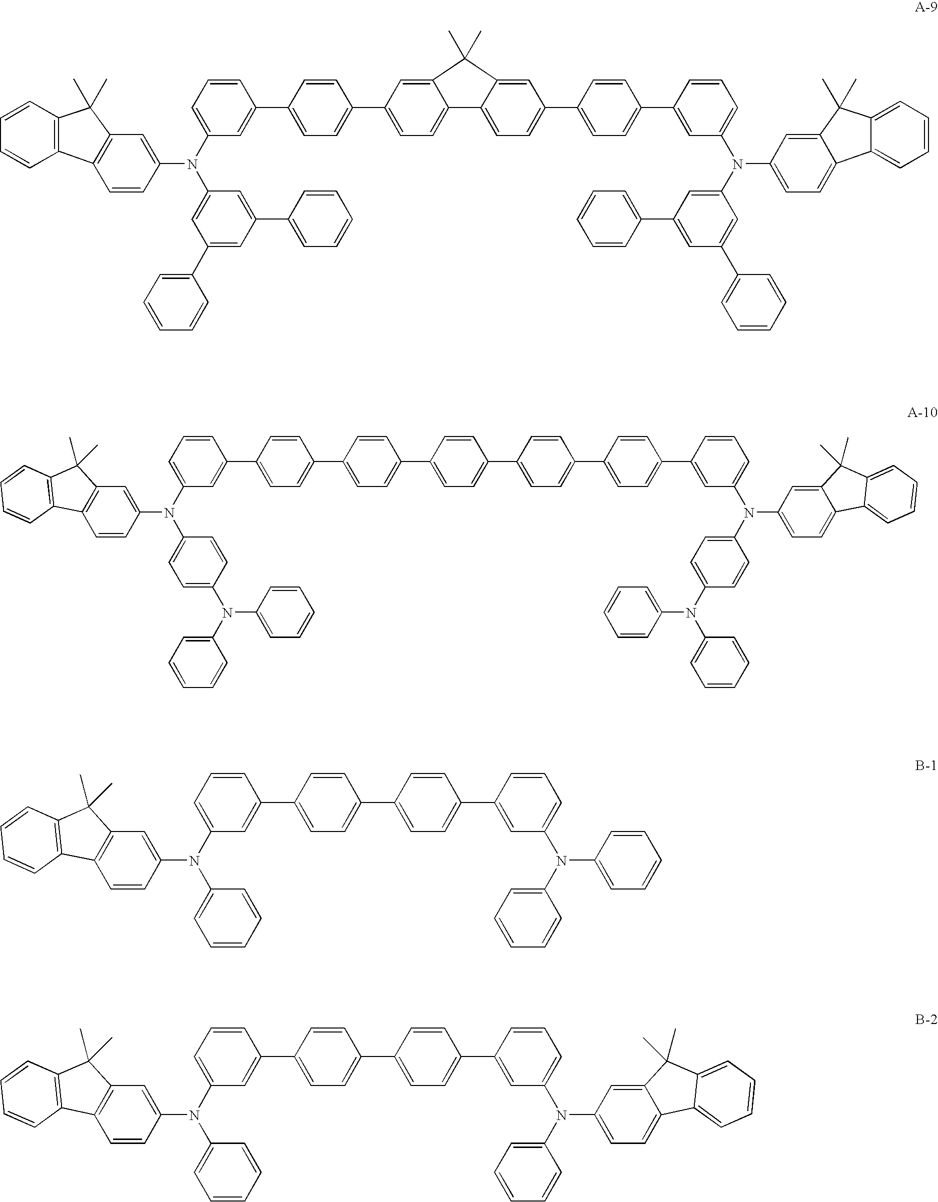

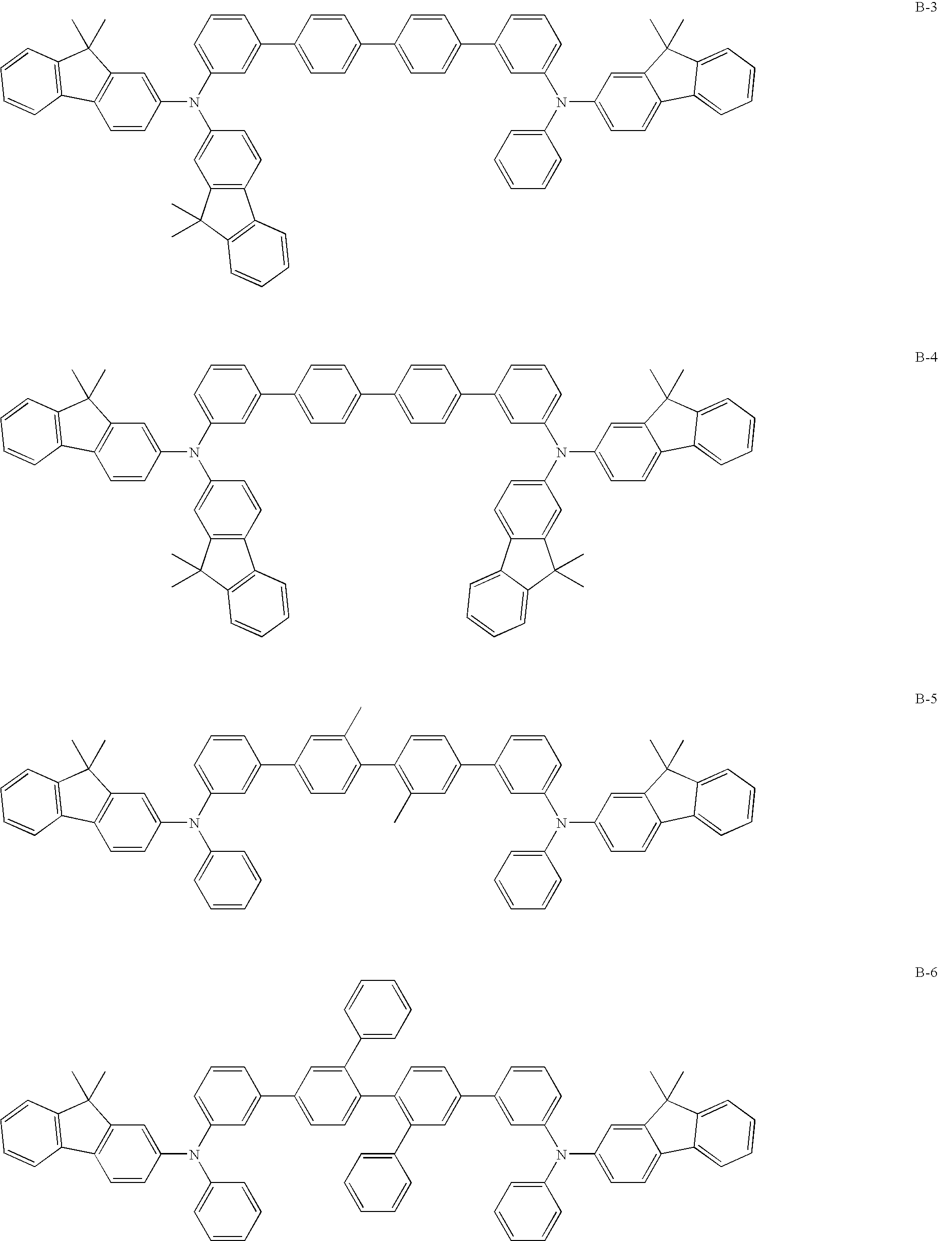

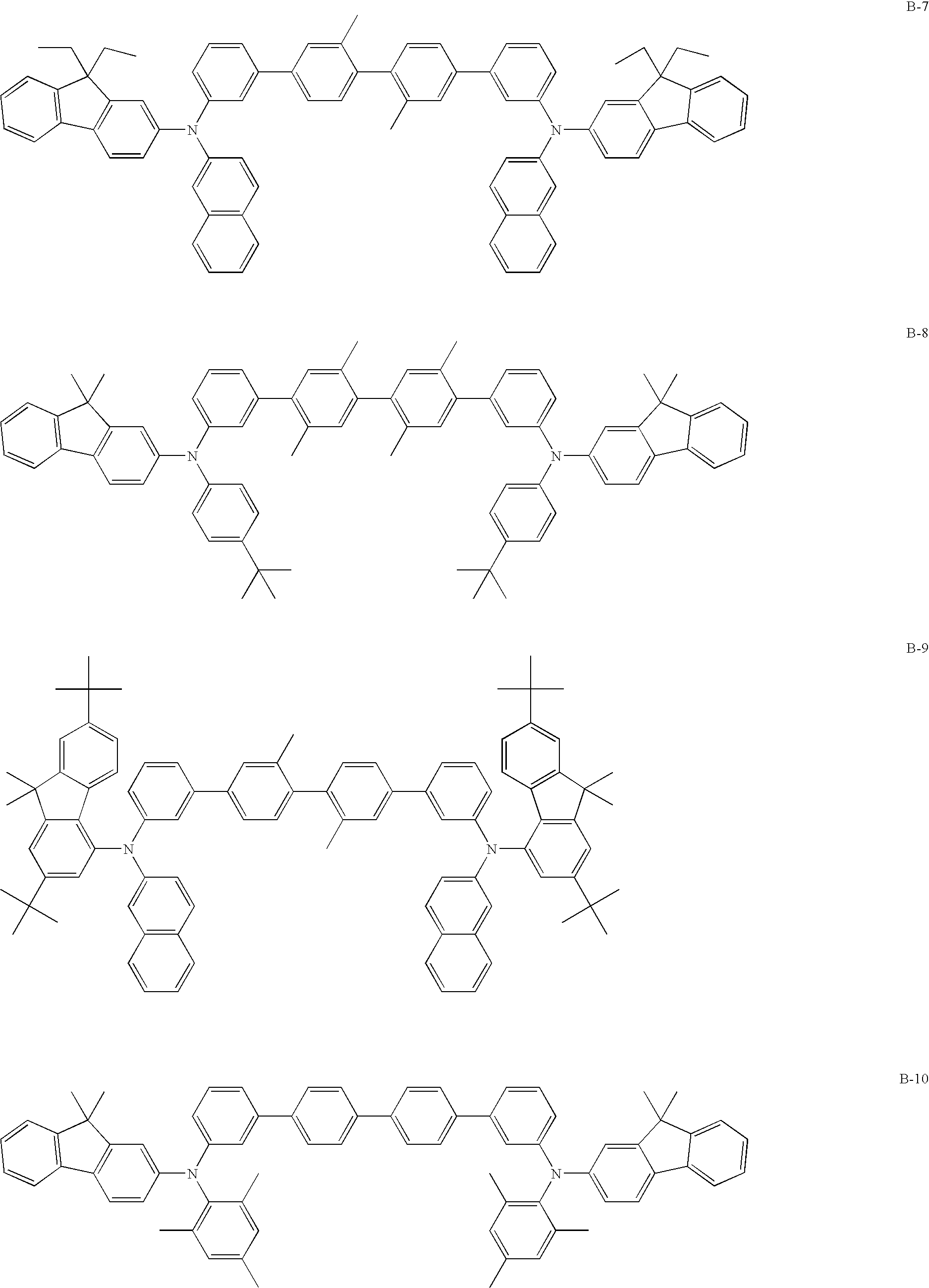

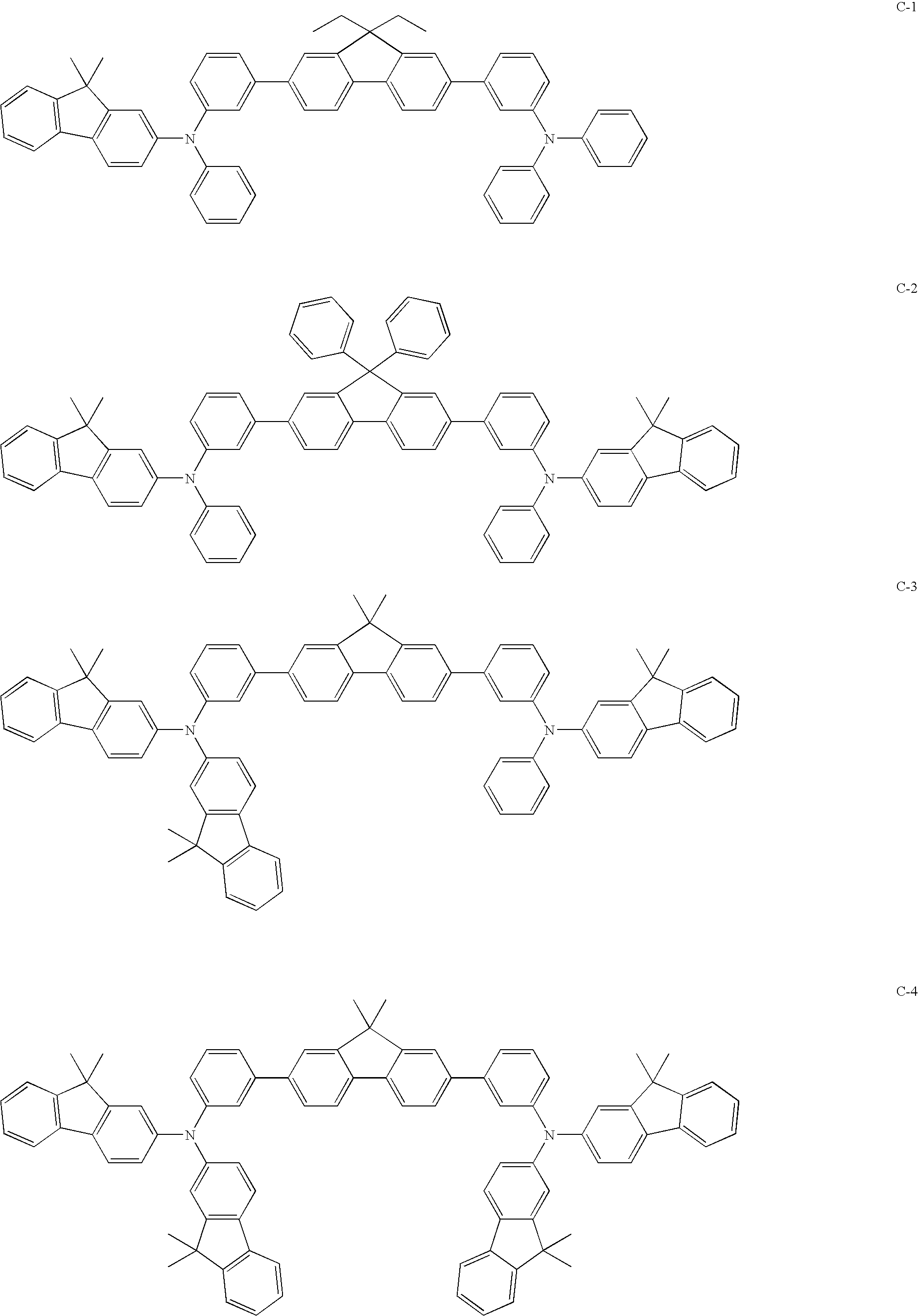

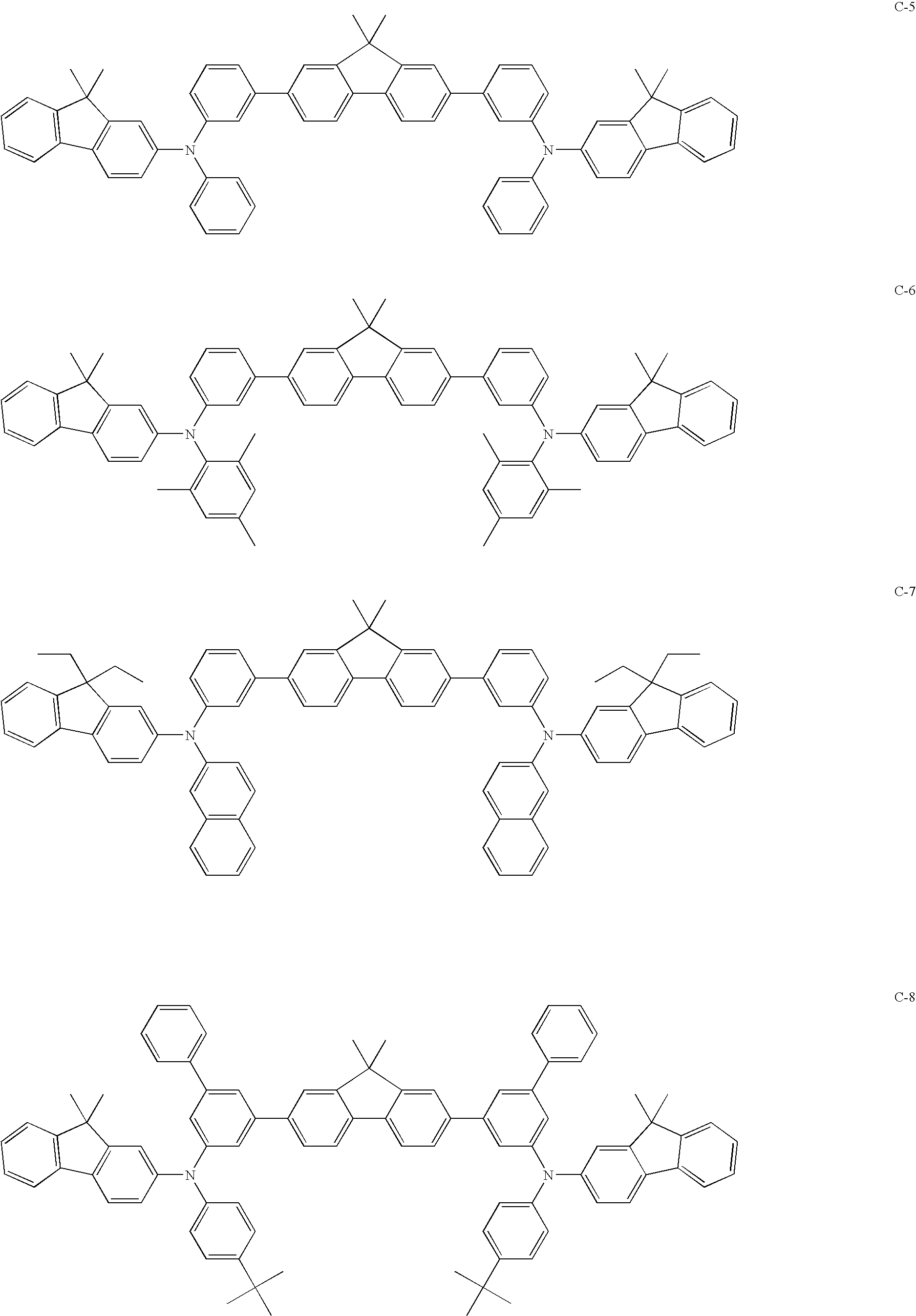

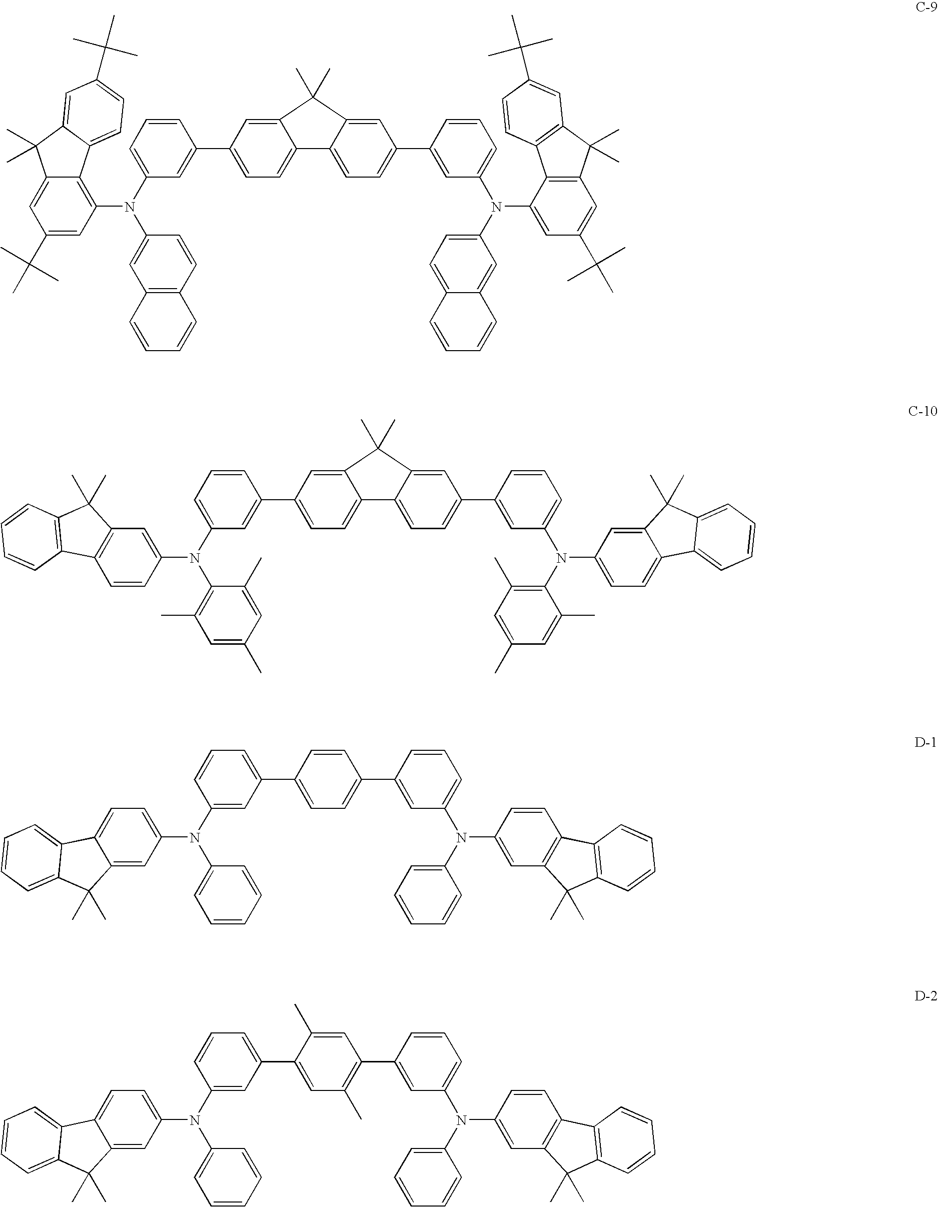

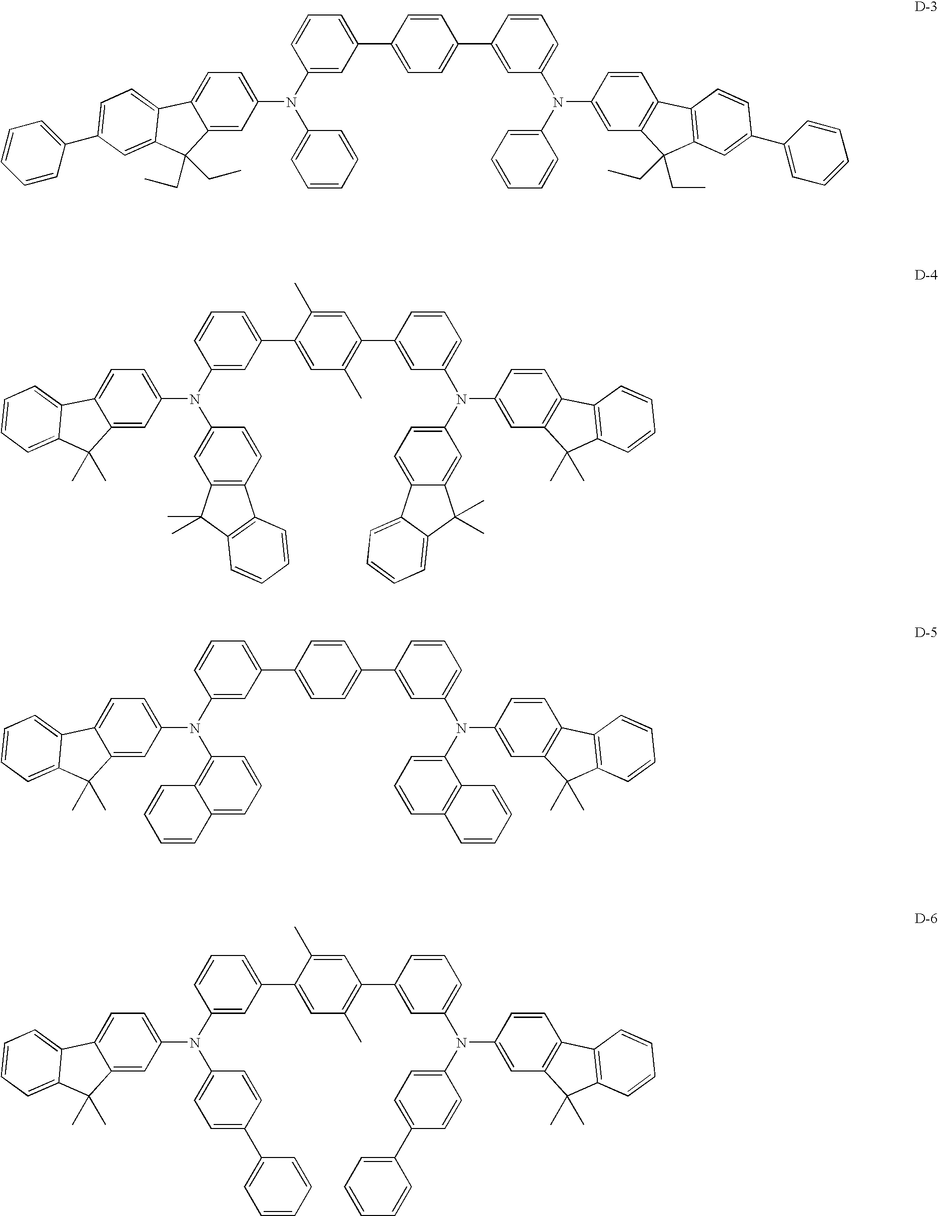

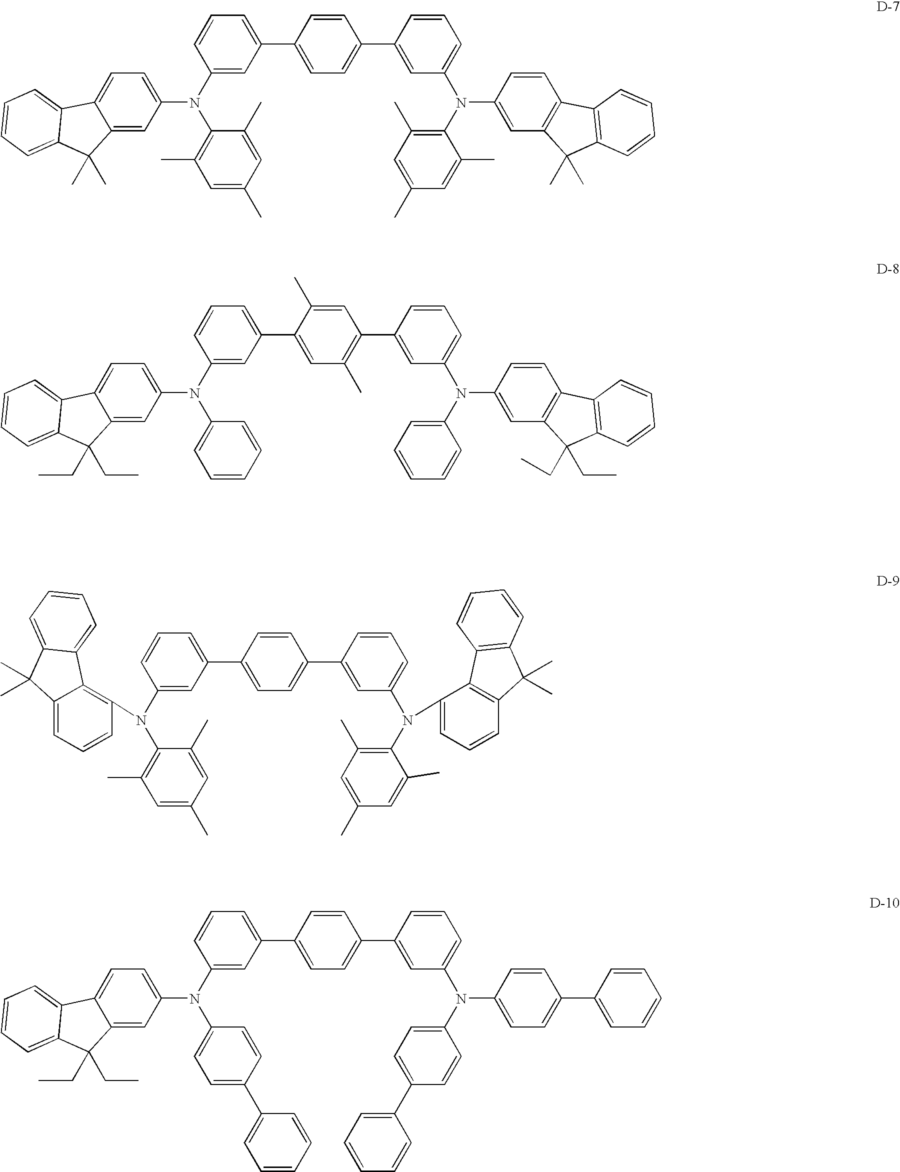

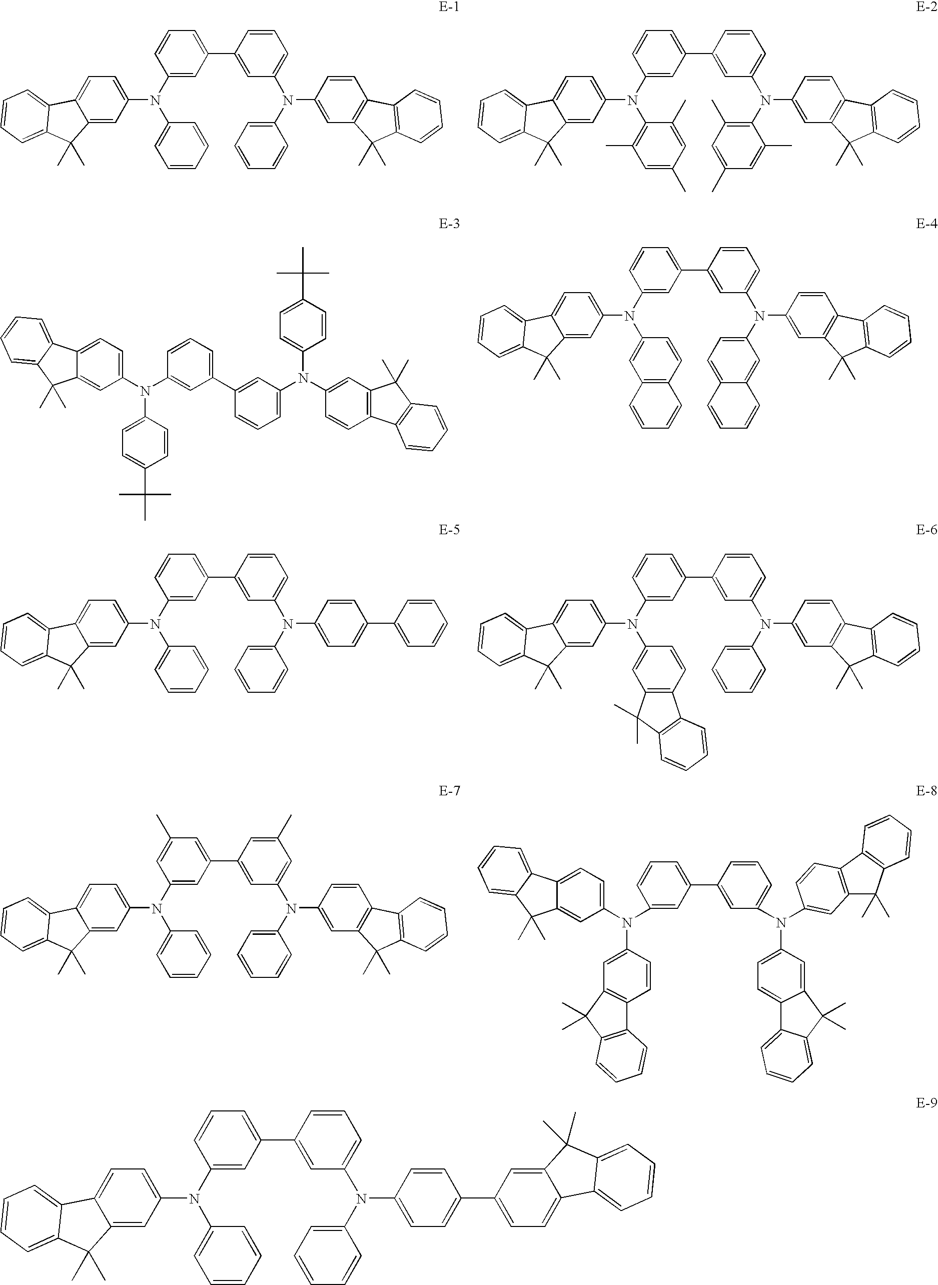

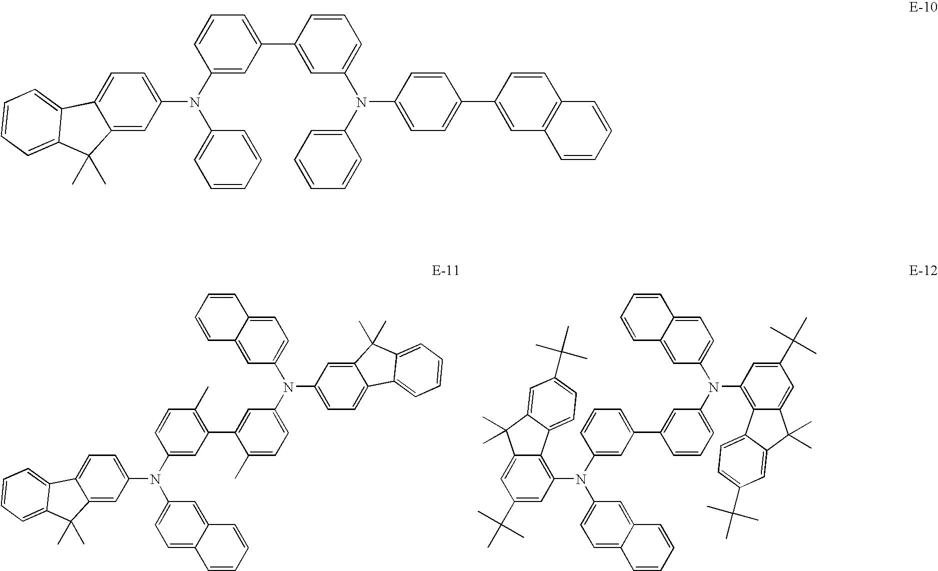

The compound represented by the general formula (1) of the present invention can be synthesized by a generally known method. The compound can be obtained by a synthesis method such as a Suzuki coupling method using a palladium catalyst (for example, Chem. Rev. 1995, 95, 2457-2483) or a Yamamoto method using a nickel catalyst (for example, Bull. Chem. Soc. Jpn. 51, 2091, 1978).

The compound represented by the general formula (1) of the present invention is a compound superior to any conventional compound in electron transport property, light emission property, and durability. In addition, the compound is useful in a layer containing an organic compound of an organic light-emitting device, in particular, each of a hole transport layer and a light-emitting layer. In addition, a layer formed by, for example, a vacuum deposition method or a solution application method hardly undergoes, for example, crystallization, and has excellent stability in time-dependent change.

Next, an organic light-emitting device of the present invention will be described in detail.

The organic light-emitting device of the present invention has at least a pair of electrodes composed of an anode and a cathode, and one or a plurality of layers each containing an organic compound, the one or plurality of layers being interposed between the pair of electrodes, wherein, at least one layer of the one or plurality of layers each containing an organic compound contains at least one kind of a compound represented by the general formula (1).

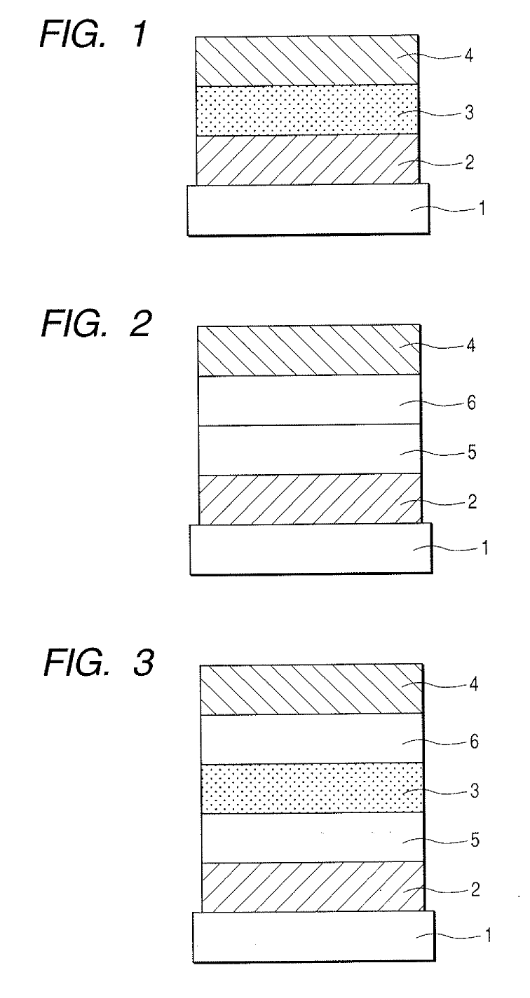

FIGS. 1 to 5 each show a preferable example of the organic light-emitting device of the present invention.

First, the reference numerals of the respective drawings will be described. Reference numeral 1 represents a substrate; 2, an anode; 3, a light-emitting layer; 4, a cathode; 5, a hole transport layer; 6, an electron transport layer; 7, a hole injection layer; and 8, a hole/exciton blocking layer.

FIG. 1 is a sectional view showing an example of the organic light-emitting device of the present invention. FIG. 1 shows a constitution in which the anode 2, the light-emitting layer 3, and the cathode 4 are sequentially provided onto the substrate 1. The light-emitting device to be used here is useful in a case where the device itself has a hole-transporting property, an electron-transporting property, and light-emitting property alone or a case where compounds having respective properties are used as a mixture.

FIG. 2 is a sectional view showing another example of the organic light-emitting device of the present invention. FIG. 2 shows a constitution in which the anode 2, the hole transport layer 5, the electron transport layer 6, and the cathode 4 are sequentially provided onto the substrate 1. In this case, a material having one or both of hole-transporting property and electron-transporting property is used as a light-emitting substance in each layer. This case is useful when the device is used in combination with a mere hole-transporting substance or electron-transporting substance having no light-emitting properties. In addition, in this case, a light-emitting layer 3 is composed of the hole transport layer 5 or the electron transport layer 6.

FIG. 3 is a sectional view showing another example of the organic light-emitting device of the present invention. FIG. 3 shows a constitution in which the anode 2, the hole transport layer 5, the light-emitting layer 3, the electron transport layer 6, and the cathode 4 are sequentially provided onto the substrate 1. This constitution separates a carrier-transporting function and a light-emitting function. In addition, the device can be formed by timely using a compound having a hole-transporting property, a compound having an electron-transporting property, and a compound having a light-emitting property in combination, so that the degree of freedom in selection of materials extremely increases. In addition, various compounds different from each other in emission wavelength can be used. As a result, the range of luminescent colors can be widened. Furthermore, an emission efficiency can be improved by effectively trapping each carrier or exciton in the central light-emitting layer 3.

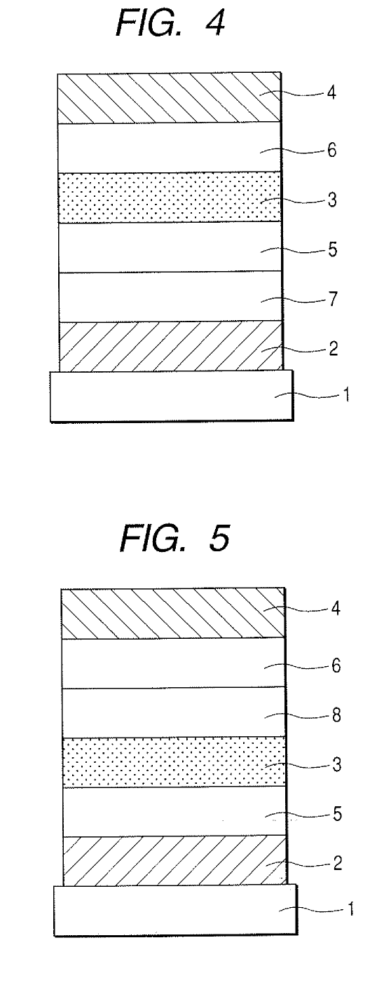

FIG. 4 is a sectional view showing another example in the organic light-emitting device of the present invention. FIG. 4 shows a constitution different from that shown in FIG. 3 in that the hole injection layer 7 is inserted on the side of the anode 2. The layer has an improved effect on adhesiveness between the anode 2 and the hole transport layer 5 or on hole injection property, and is effective for a reduction in voltage at which the device is driven.

FIG. 5 is a sectional view showing another example in the organic light-emitting device of the present invention. FIG. 5 shows a constitution different from that shown in FIG. 3 in that a layer (hole/exciton blocking layer 8) for inhibiting the escape of a hole or an exciton toward the side of the cathode 4 is inserted between the light-emitting layer 3 and the electron transport layer 6. The constitution is effective for an improvement in luminous efficiency when a compound having an extremely high ionization potential is used in the hole/exciton blocking layer 8.

The device constitutions shown in FIGS. 1 to 5 are merely very basic constitutions, and the constitution of an organic light-emitting device using the compound of the present invention is not limited to these constitutions. The device may adopt any one of various layer constitutions. For example, an insulating layer may be provided onto an interface between an electrode and an organic layer. Alternatively, an adhesive layer or an interference layer may be provided. In addition, a hole transport layer is constituted of two layers of different ionization potential.

The compound represented by the general formula (1) to be used in the present invention can be used in any one of the forms shown in FIGS. 1 to 5.

In the present invention, a compound represented by the general formula (1) is used particularly as a component of a light-emitting layer; a conventionally known, low-molecular-weight-based or polymer-based hole transport compound, luminescent compound, electron transport compound, or the like can be used together with the compound as required.

In an organic light-emitting device of the present invention, a layer containing an amine compound represented by the general formula (1) and a layer composed of another organic compounds each generally form a thin film by a vacuum deposition method, an ionization deposition method, a sputtering method, or plasma. Alternatively, each layer was dissolved into an appropriate solvent to form a thin film by a known application method such as spin coating, a dipping method, a casting method, an LB method, an inkjet method, or the like. In particular, in the case where a thin film is formed by an application method, the film may also be formed in combination with an appropriate binder resin.

The binder resin may be selected from a wide variety of binder resin. Examples of the binder resin include, but not limited to, a polyvinyl carbazole resin, a polycarbonate resin, a polyester resin, a polyarylate resin, a polystyrene resin, an ABS resin, a polybutadine resin, a polyurethane resin, an acrylic resin, a methacrylic resin, a butyral resin, a polyvinyl acetal resin, a polyamide resin, a polyimide resin, a polyethylene resin, a polyethersulfone resin, a diallyl phthalate resin, a phenol resin, an epoxy resin, a silicone resin, a polysulfone resin, and a urea resin. Each of those resins may be used alone, or one or more of them may be mixed as a copolymer. Further, an additive such as a known plasticizer, antioxidant, or ultraviolet absorber may be used together if required.

An anode material preferably has as large a work function as possible. Examples of the anode material include a metal such as gold, platinum, silver, copper, nickel, palladium, cobalt, selenium, vanadium, or tungsten. In addition, each of alloys thereof and metal oxides such as tin oxide, zinc oxide, indium oxide, indium tin oxide (ITO), and indium zinc oxide may be used. Further, a conductive polymer such as polyaniline, polypyrrole, polythiophene, or polyphenylene sulfide may also be used. Each of those electrode substances may be used alone, or two or more of them may be used in combination. Further, an anode may adopt a single layer construction or a multilayer construction.

On the other hand, a cathode material preferably has a small work function. Examples of the cathode material which can be used include: a metal such as lithium, sodium, potassium, calcium, magnesium, aluminum, indium, ruthenium, titanium, manganese, yttrium, silver, lead, tin, or chromium; and an alloy composed of a plurality of metals such as lithium-indium, sodium-potassium, magnesium-silver, aluminum-lithium, aluminum-magnesium, or magnesium-indium. A metal oxide such as indium tin oxide (ITO) may also be used. Each of those electrode substances may be used alone, or two or more of them may be used in combination. Further, a cathode may adopt a single layer construction or a multilayer construction.

Further, at least either of an anode or a cathode may desirably be transparent or semi-transparent.

A substrate to be used in the present invention is not particularly limited; provided that an opaque substrate such as a metallic substrate or a ceramic substrate, or a transparent substrate such as glass, quartz, or a plastic sheet is used. In addition, a luminescent color can be controlled by using a color filter film, a fluorescent color conversion filter film, a dielectric reflective film, or the like as the substrate.

In addition, a luminescent color can be controlled by using a color filter film, a fluorescent color conversion filter film, a dielectric reflective film, or the like as the substrate. Alternatively, a device can be produced by producing a thin film transistor (TFT) on a substrate and by connecting the TFT to the substrate.

In addition, with regard to the direction in which light is extracted from the device, both a bottom emission constitution (constitution in which light is extracted from a substrate side) and a top emission constitution (constitution in which light is extracted from the side opposite to the substrate) are available.

It should be noted that the produced device may be provided with a protective layer or a sealing layer for the purpose of preventing the device from contacting with, for example, oxygen or moisture. Examples of the protective layer include: an inorganic material film such as a diamond thin film, a metal oxide, or a metal nitride; a polymer film such as a fluorine resin, polyparaxylene, polyethylene, a silicone resin, or a polystyrene resin; and a photocurable resin. In addition, the device itself may be covered with, for example, glass, a gas impermeable film, or a metal, and packaged with an appropriate sealing resin.

An ionization potential as specified in the present invention is defined as energy needed for an electron at the highest occupied molecular orbital (HOMO) level of a compound to be released to a vacuum level. Meanwhile, an electron affinity is defined as energy with which an electron at a vacuum level drops to the lowest unoccupied molecular orbital (LUMO) level of a substance to stabilize.

An ionization potential can be directly measured by ultraviolet photoelectron spectroscopy (UPS) or by using a low-energy electron spectrometer (measuring instrument name AC-1, AC-2, or AC-3 manufactured by RIKENKEIKI CO., LTD). Alternatively, the ionization potential can be determined from, for example, the measurement of an oxidation potential by a cyclic voltammetry method.

In the present invention, a value for an ionization potential was defined as a value measured with an AC-1 manufactured by RIKENKEIKI CO., LTD.

An electron affinity is defined by the following expression:

(Electron affinity)=(Ionization potential)−(Band gap).

The band gap can be measured by, for example, a method involving: depositing an organic compound from the vapor onto glass to provide a deposited film having a thickness of about 50 nm; measuring the absorption spectrum of the deposited film; and converting a wavelength Y (nm) of an absorption edge of the spectrum into X (eV).

X can be determined by a conversion expression “X=1,240/Y”.

The electron affinity can be determined also from the measurement of a reduction potential by a cyclic voltammetry method.

A method of determining an electron affinity adopted in the present invention involved calculating the electron affinity from a measured value of a band gap by light absorption and the above ionization potential. A spectrophotometer U-3010 (manufactured by Hitachi High-Technologies Corporation) was used for measuring an absorption spectrum.

Hereinafter, the present invention will be described more specifically by way of the following Examples. However, the present invention is not limited to these Examples.

### EXAMPLE 1

### Synthesis of Exemplified Compound C-5

a) Synthesis of intermediate compound

Compound (1-1) can be produced by an Ullmann reaction by using N-(9,9-dimethyl-fluorene-2-yl)-N-phenyl-amine and 3-bromoiodobenzene as raw materials.

### b) Synthesis of Exemplified Compound C-5

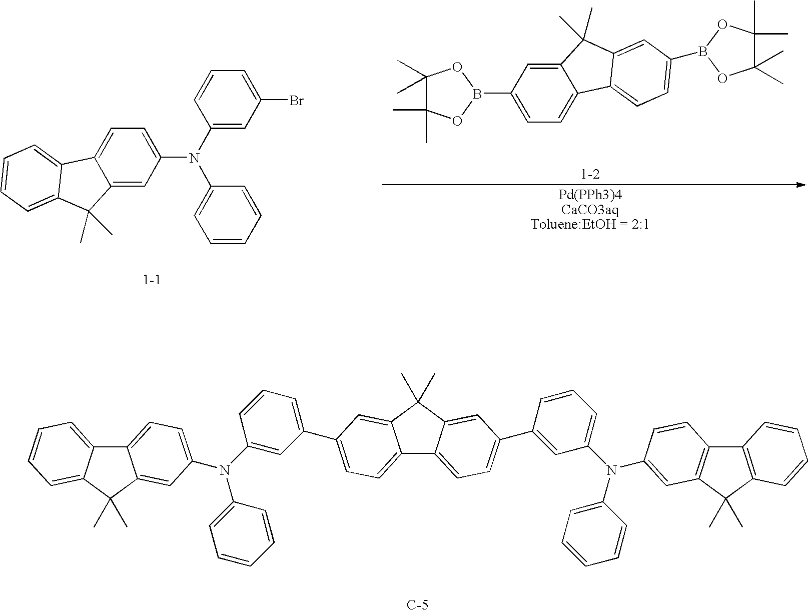

A 500-ml three-necked flask was prepared. 3.0 g (6.74 mmol) of Compound (1-2) were loaded into the flask. Further, 7.42 g (16.9 mmol) of Compound (1-1) were loaded into the flask. Further, 140 ml of toluene and 70 ml of ethanol were loaded into the flask, and an aqueous solution prepared by dissolving 3 g of calcium carbonate in 30 ml of water was dropped while the mixture was stirred in a nitrogen atmosphere at room temperature. Next, 0.39 g (0.34 mmol) of tetrakis(triphenylphosphine)palladium(0) was added. After the resultant had been stirred at room temperature for 30 minutes, the temperature of the resultant was increased to 70° C., and then the resultant was stirred for 6 hours. After the reaction, an organic layer was extracted with toluene, dried with anhydrous sodium sulfate, and purified with a silica gel column (mixed developing solvent of hexane and toluene), whereby 4.42 g of Exemplified Compound C-5 (white crystal) were obtained (72% yield). Matrix-assisted laser desorption/ionization time-of-flight mass spectrometry (MALDI-TOF MS) confirmed that the M+ of the compound was 912.4. Thermogravimetry (TG) and differential thermal analysis (DTA) were simultaneously performed to confirm that the compound had a melting point of 282° C. It was confirmed that the compound had an ionization potential of 5.46 eV, an electron affinity of 2.24 eV, and a band gap of 3.22 eV.

### EXAMPLE 2

### Synthesis of Exemplified Compound B-2

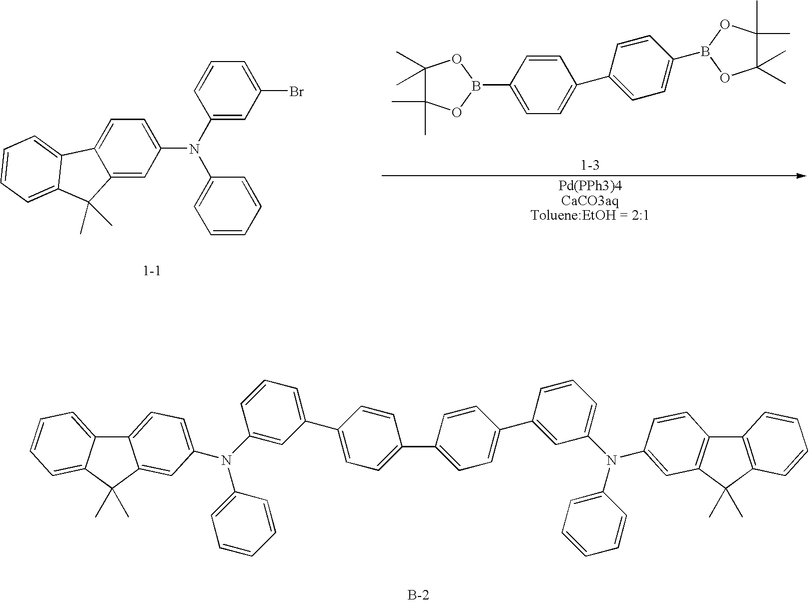

A 300-ml three-necked flask was prepared. 1.8 g (4.4 mmol) of Compound (1-3) were loaded into the flask. Further, 4.3 g (9.7 mmol) of Compound (1-1) were loaded into the flask. Further, 100 ml of toluene and 50 ml of ethanol were loaded into the flask, and an aqueous solution prepared by dissolving 2 g of calcium carbonate in 20 ml of water was dropped while the mixture was stirred in a nitrogen atmosphere at room temperature. Next, 0.25 g (0.22 mmol) of tetrakis(triphenylphosphine)palladium(0) was added. After the resultant had been stirred at room temperature for 30 minutes, the temperature of the resultant was increased to 70° C., and then the resultant was stirred for 9 hours. After the reaction, an organic layer was extracted with toluene, dried with anhydrous sodium sulfate, and purified with a silica gel column (mixed developing solvent of hexane and toluene), whereby 1.4 g of Exemplified Compound B-2 (white crystal) were obtained (74% yield). Matrix-assisted laser desorption/ionization time-of-flight mass spectrometry (MALDI-TOF MS) confirmed that the M+ of the compound was 872.4. Thermogravimetry (TG) and differential thermal analysis (DTA) were simultaneously performed to confirm that the compound had a melting point of 260° C. It was confirmed that the compound had an ionization potential of 5.46 eV, an electron affinity of 2.24 eV, and a band gap of 3.22 eV.

### EXAMPLE 3

### Synthesis of Exemplified Compound D-2

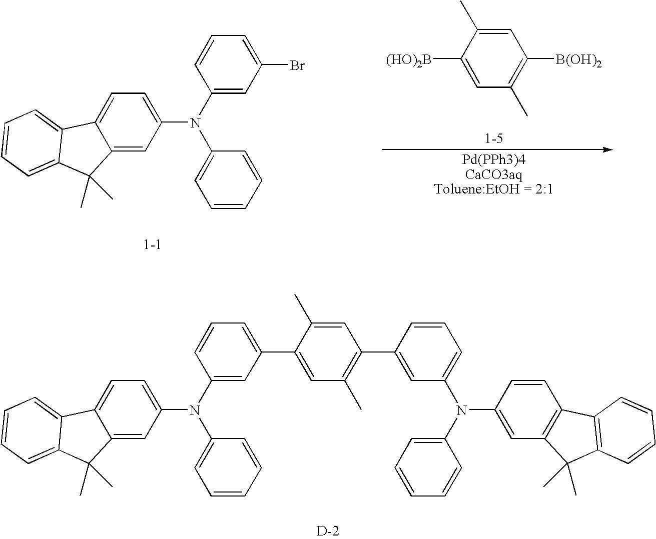

A 300-ml three-necked flask was prepared. 0.41 g (2.1 mmol) of Compound (1-5) were loaded into the flask. Further, 2.0 g (4.6 mmol) of Compound (1-1) were loaded into the flask. Further, 100 ml of toluene and 50 ml of ethanol were loaded into the flask, and an aqueous solution prepared by dissolving 2 g of calcium carbonate in 20 ml of water was dropped while the mixture was stirred in a nitrogen atmosphere at room temperature. Next, 0.13 g (0.11 mmol) of tetrakis(triphenylphosphine)palladium(0) was added. After the resultant had been stirred at room temperature for 30 minutes, the temperature of the resultant was increased to 70° C., and then the resultant was stirred for 8 hours. After the reaction, an organic layer was extracted with toluene, dried with anhydrous sodium sulfate, and purified with a silica gel column (mixed developing solvent of hexane and toluene), whereby 1.2 g of Exemplified Compound D-2 (white crystal) were obtained (68% yield). Matrix-assisted laser desorption/ionization time-of-flight mass spectrometry (MALDI-TOF MS) confirmed that the M+ of the compound was 824.4. Thermogravimetry (TG) and differential thermal analysis (DTA) were simultaneously performed to confirm that the compound had a melting point of 243° C. It was confirmed that the compound had an ionization potential of 5.50 eV, an electron affinity of 2.30 eV, and a band gap of 3.20 eV.

### EXAMPLE 4

### Synthesis of Exemplified Compound E-5

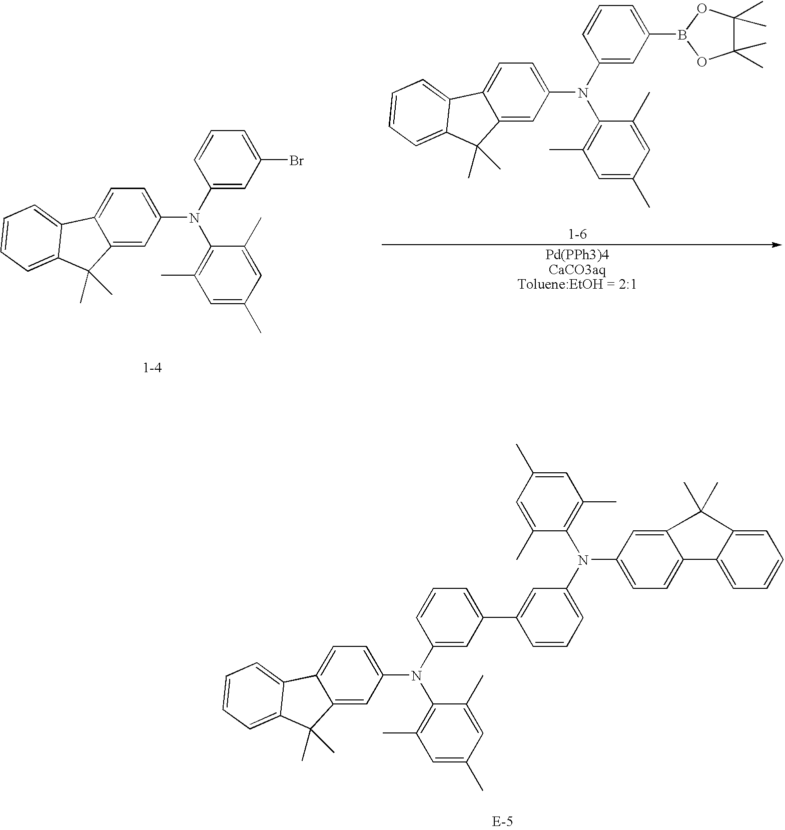

A 300-ml three-necked flask was prepared. 1.2 g (2.5 mmol) of Compound (1-6) were loaded into the flask. Further, 1.3 g (2.5 mmol) of Compound (1-4) were loaded into the flask. Further, 100 ml of toluene and 50 ml of ethanol were loaded into the flask, and an aqueous solution prepared by dissolving 2 g of calcium carbonate in 20 ml of water was dropped while the mixture was stirred in a nitrogen atmosphere at room temperature. Next, 0.29 g (0.25 mmol) of tetrakis(triphenylphosphine)palladium(0) was added. After the resultant had been stirred at room temperature for 30 minutes, the temperature of the resultant was increased to 80° C., and then the resultant was stirred for 10 hours. After the reaction, an organic layer was extracted with toluene, dried with anhydrous sodium sulfate, and purified with a silica gel column (mixed developing solvent of hexane and toluene), whereby 1.7 g of Exemplified Compound E-5 (white crystal) were obtained (82% yield). Matrix-assisted laser desorption/ionization time-of-flight mass spectrometry (MALDI-TOF MS) confirmed that the M+ of the compound was 804.4. Thermogravimetry (TG) and differential thermal analysis (DTA) were simultaneously performed to confirm that the compound had a melting point of 228° C. It was confirmed that the compound had an ionization potential of 5.40 eV, an electron affinity of 2.16 eV, and a band gap of 3.24 eV.

### EXAMPLE 5

### Synthesis of Exemplified Compound E-1

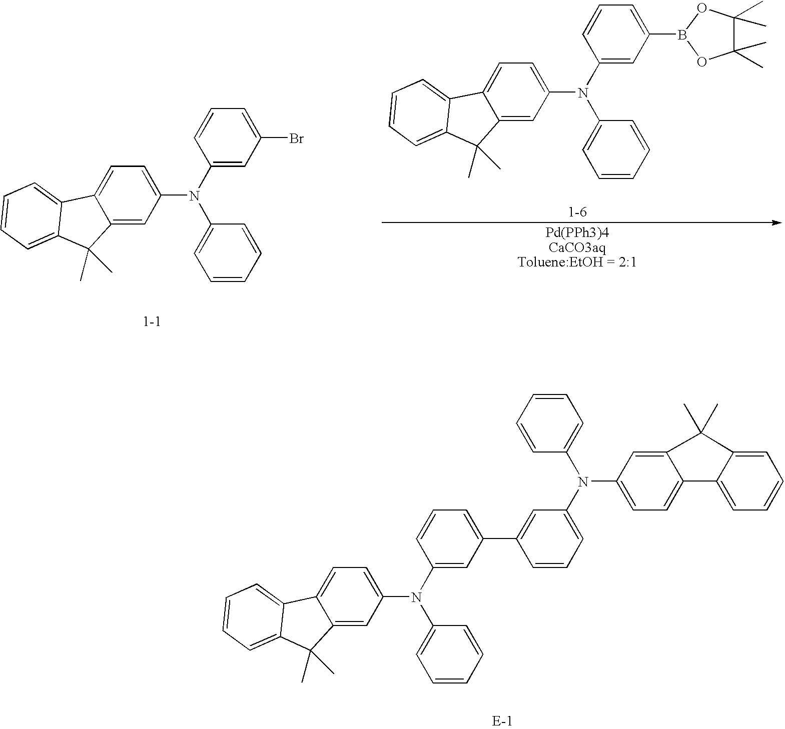

A 100-ml three-necked flask was prepared. 0.40 g (0.82 mmol) of Compound (1-7) were loaded into the flask. Further, 0.36 g (0.82 mmol) of Compound (1-1) were loaded into the flask. Further, 50 ml of toluene and 25 ml of ethanol were loaded into the flask, and an aqueous solution prepared by dissolving 1 g of calcium carbonate in 5 ml of water was dropped while the mixture was stirred in a nitrogen atmosphere at room temperature. Next, 0.12 g (0.1 mmol) of tetrakis(triphenylphosphine)palladium(0) was added. After the resultant had been stirred at room temperature for 30 minutes, the temperature of the resultant was increased to 70° C., and then the resultant was stirred for 5 hours. After the reaction, an organic layer was extracted with toluene, dried with anhydrous sodium sulfate, and purified with a silica gel column (mixed developing solvent of hexane and toluene), whereby 0.44 g of Exemplified Compound E-1 (white crystal) were obtained (75% yield). Matrix-assisted laser desorption/ionization time-of-flight mass spectrometry (MALDI-TOF MS) confirmed that the M+ of the compound was 720.3. Thermogravimetry (TG) and differential thermal analysis (DTA) were simultaneously performed to confirm that the compound had a melting point of 220° C. It was confirmed that the compound had an ionization potential of 5.46 eV, an electron affinity of 2.24 eV, and a band gap of 3.22 eV.

### EXAMPLE 6

An organic light-emitting device having a structure shown in FIG. 3 was produced by the following method.

Indium tin oxide (ITO) was formed by a sputtering method into a film having a thickness of 120 nm to serve as the anode 2 on a glass substrate as the substrate 1, and the resultant was used as a transparent, conductive supporting substrate. The substrate was subjected to ultrasonic cleaning with acetone and isopropyl alcohol (IPA) sequentially. Then, the substrate was subjected to boiling cleaning with IPA, followed by drying. Further, the substrate was subjected to UV/ozone cleaning. The resultant was used as a transparent, conductive supporting substrate.

Exemplified Compound C-5 was formed by a vacuum deposition method into a film having a thickness of 20 nm to serve as the hole transport material. The film was formed under conditions including: a degree of vacuum upon deposition of 1.0×10⁻⁴ Pa; and a film formation rate of 0.1 nm/sec.

Next, Compound 2-1 shown below as a first compound and Compound 3-1 shown below as a second compound were co-deposited from the vapor (at a weight ratio of 90:10) onto the hole transport layer 5 to provide the light-emitting layer 3 having a thickness of 20 nm. The layer was formed under conditions including: a degree of vacuum upon deposition of 1.0×10⁻⁴ Pa; and a film formation rate of 0.1 nm/sec.

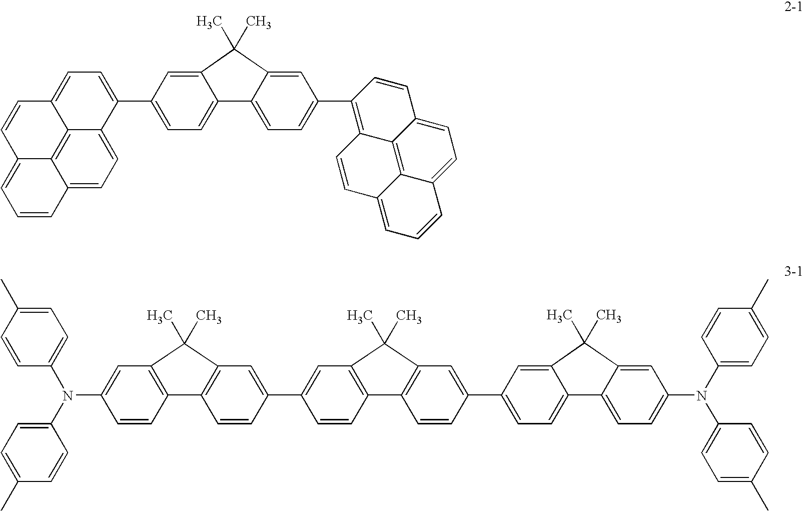

Compound 2-1 (ionization potential: 5.67 eV, electron affinity: 2.72 eV, band gap: 2.95 eV)

Compound 3-1 (ionization potential: 5.31 eV, electron affinity: 2.39 eV, band gap: 2.92 eV)

Further, bathophenanthroline (BPhen) was formed by a vacuum deposition method into a film having a thickness of 40 nm to serve as the electron transport layer 6. The film was formed under conditions including: a degree of vacuum upon deposition of 1.0×10⁻⁴ Pa; and a film formation rate of 0.2 to 0.3 nm/sec.

Next, a metal layer film having a thickness of 0.5 nm was formed by a vacuum deposition method on the foregoing organic layer by using a deposition material composed of an aluminum-lithium alloy (lithium concentration: 1% by atom). Further, an aluminum film having a thickness of 150 nm was provided by a vacuum deposition method. Thus, an organic light-emitting device using the aluminum-lithium alloy film as an electron injection electrode (cathode 4) was produced. The films were each formed under conditions including: a degree of vacuum upon deposition of 1.0×10⁻⁴ Pa; and a film formation rate of 1.0 to 1.2 nm/sec.

The resultant organic EL device was covered with a protective glass plate in a dry air atmosphere and sealed with an acrylic resin-based adhesive in order that the device might be prevented from deteriorating owing to the adsorption of moisture.

A voltage of 5.0 V was applied to the thus-obtained device by using the ITO electrode (anode 2) as a positive electrode and the Al electrode (cathode 4) as a negative electrode. As a result, the device was observed to emit blue light having a luminance of 2,800 cd/m² and an emission center wavelength of 458 nm.

Further, a voltage was applied for 100 hours while a current density was kept at 30 mA/cm² under a nitrogen atmosphere. As a result, an initial luminance of 1,720 cd/m² reduced to 1,280 cd/m² in 100 hours (the result is shown in Table 1 as a list).

### EXAMPLES 7 TO 10

Devices were each produced in the same manner as in Example 6 except that Exemplified Compound B-2, D-2, E-5, or E-1 was used instead of Exemplified Compound C-5, and were each evaluated in the same manner as in Example 6 (the results are shown in Table 1 as a list).

### COMPARATIVE EXAMPLE 1

A device was produced in the same manner as in Example 1 except that Comparative Compound 4-1 shown below was used instead of Exemplified Compound C-5, and was evaluated in the same manner as in Example 1. The device was observed to emit blue light having a luminance of 1,800 cd/m² and a wavelength of 458 nm at an applied voltage of 5.0 V. Further, a voltage was applied for 100 hours while a current density was kept at 30 mA/cm² under a nitrogen atmosphere. As a result, an initial luminance of 1,250 cd/m² reduced to 500 cd/m² in 100 hours (the result is shown in Table 1 as a list).

TABLE 1

| | |
|---|---|
| | 4-1 |
| 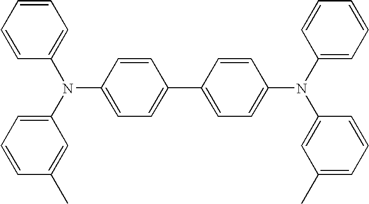 | |

| | | Initial | | Duration | | |
|---|---|---|---|---|---|---|
| | | | | (Current | | Luminance |
| | | Applied | | density) | Initial | after |
| | Compound | voltage | Luminance | 30.0 | luminance | 100 hours |
| | No. | (V) | (cd/m²) | mA/cm² | (cd/m²) | (cd/m²) |
| Example | C-5 | 5 | 2800 | | 1720 | 1280 |
| 6 | | | | | | |
| 7 | B-2 | 5 | 2830 | | 1750 | 1350 |
| 8 | D-2 | 5 | 2260 | | 1590 | 980 |
| 9 | E-5 | 5 | 2900 | | 1800 | 1360 |
| 10 | E-1 | 5 | 2720 | | 1620 | 1160 |
| Comparative | 4-1 | 5 | 1800 | | 1250 | 500 |
| Example | | | | | | |
| 1 | | | | | | |

Compound 4-1 (ionization potential: 5.50 eV, electron affinity: 2.35 eV, band gap: 3.15 eV)

Table 1 below shows the list of the summary of the results of investigation on Examples 6 to 10 and Comparative Example 1.

In addition, values and evaluation represented by A to H of each of Exemplified Compounds C-5, B-2, D-2, E-5, and E-1, and Compound 4-1 used in Examples 6 to 10 and Comparative Example 1 are shown below.

TABLE 2

| | Com- | | | | | | | | |
|---|---|---|---|---|---|---|---|---|---|
| | pound | | | | | | | | |
| | No. | A | B | C | D | E | F | G | H |
| Example | C-5 | 5.46 | 2.24 | 3.22 | 0.48 | 0.27 | 2800 | 74 | a |
| 6 | | | | | | | | | |
| 7 | B-2 | 5.46 | 2.24 | 3.22 | 0.48 | 0.27 | 2830 | 77 | a |
| 8 | D-2 | 5.5 | 2.30 | 3.20 | 0.42 | 0.25 | 2260 | 61 | b |
| 9 | E-5 | 5.4 | 2.16 | 3.24 | 0.56 | 0.29 | 2900 | 75 | a |
| 10 | E-1 | 5.46 | 2.24 | 3.22 | 0.48 | 0.27 | 2720 | 72 | a |
| Compar- | 4-1 | 5.50 | 2.35 | 3.15 | 0.37 | 0.20 | 1800 | 40 | c |
| ative | | | | | | | | | |
| Example | | | | | | | | | |
| 1 | | | | | | | | | |

A: Absolute value for an ionization potential
B: Absolute value for an electron affinity
C: A − B (value for a band gap)
D: 2.72 (absolute value of the electron affinity of Compound 2-1 used as a host of a light-emitting layer) − B
E: C − 2.95 (value of the band gap of Compound 2-1 used as a host of a light-emitting layer)
F: Initial luminance at an applied voltage of 5 V (cd/m2)
G: Value of a luminance after 100 hours represented in a percentage unit when a current density is kept at 30 mA/cm² and an initial luminance is set to 1.
H: a . . . F is 2,700 or more, and G is 70 or more
b . . . F is 2,100 or more and less than 2,700, and G is 50 or more and less than 70
c . . . F is less than 2,100, and G is less than 50

Paying attention to the values for B and D described in Table 2 found that an emission luminance (F) tended to be larger as a value obtained by subtracting the absolute value for the electron affinity of a hole transport material (B) from the absolute value for the electron affinity of a host material for use in a light-emitting layer became larger. Further, it was found that, when D was 0.42 or more and B was smaller than 2.30, an increasing effect on the emission luminance was particularly significant (comparison between any one of Examples 6 to 10 and Comparative Example 1). In addition, it was also found that the emission luminance of Comparative Example 1 was smaller than that of any one of Examples 6 to 10. Those findings show that an improvement in electron blocking property in a hole transport layer can improve an emission luminance. That is, it was found that a large value for D contributed to an improvement in emission luminance. In addition, it was found from Table 2 that an increase in value for C was effective for an improvement in electron blocking property of the hole transport layer, that is, an increase in value for D. Further, it can be found from Table 2 that an improvement in electron blocking property of the hole transport layer requires the widening of a difference in band gap between the hole transport layer and a host for the light-emitting layer (E). The result of the evaluation of Comparative Example 1 for the above item H is c. In order that the result may be b or a, C must be 3.20 or more, D must be 0.42 or more, and E must be 0.25 or more. Further, it was found that E of 0.27 or more and D of 0.48 or more were more preferable in order that those effects might be exerted. In addition, such effects are considered to be particularly effective for an organic blue-light-emitting device having a large light emission energy gap among an organic red-light-emitting device, an organic green-light-emitting device, and the organic blue-light-emitting device.

### EXAMPLE 11

An organic light-emitting device having a structure shown in FIG. 4 was produced by the following method.

Indium tin oxide (ITO) was formed by a sputtering method into a film having a thickness of 120 nm to serve as the anode 2 on a glass substrate as the substrate 1, and the resultant was used as a transparent, conductive supporting substrate. The substrate was subjected to ultrasonic cleaning with acetone and isopropyl alcohol (IPA) sequentially. Then, the substrate was subjected to boiling cleaning with IPA, followed by drying. Further, the substrate was subjected to UV/ozone cleaning. The resultant was used as a transparent, conductive supporting substrate.

A chloroform solution was prepared by using Compound 1 represented by the following constitutional formula 5-1 as a hole injection material in such a manner that the concentration of the compound would be 0.1 wt %.

The solution was dropped onto the above ITO electrode, and the whole was subjected to spin coating initially at a number of revolutions of 500 RPM for 10 seconds and then at a number of revolutions of 1,000 RPM for 1 minute, whereby a film was formed. After that, the resultant was dried for 10 minutes in a vacuum oven at 80° C., whereby the solvent in the thin film was completely removed. The first hole injection layer 7 thus formed had a thickness of 11 nm.

Next, Exemplified Compound C-5 as a hole transport layer was deposited from the vapor onto the hole injection layer 7 to provide an light-emitting layer 3 having a thickness of 20 nm. The layer was formed under conditions including: a degree of vacuum upon deposition of 1.0×10⁻⁴ Pa; and a film formation rate of 0.1 nm/sec.

A light-emitting layer, an electron transport layer, and an Al electrode were each produced in the same manner as in Example 6.

A voltage of 5.0 V was applied to the thus-obtained device by using the ITO electrode (anode 2) as a positive electrode and the Al electrode (cathode 4) as a negative electrode. As a result, the device was observed to emit blue light having a luminance of 3,180 cd/m² and a center wavelength of 459 μm.

Further, a voltage was applied for 100 hours while a current density was kept at 30 mA/cm² under a nitrogen atmosphere. As a result, an initial luminance of 2,030 cd/m² reduced to 1,520 cd/m² in 100 hours.

### EXAMPLES 12 TO 15

The devices were each produced in the same manner as in Example 11 by using Compound 5-1 in a hole injection layer except that Exemplified Compound B-2, D-2, E-5, or E-1 was used in a hole transport layer instead of Exemplified Compound C-5, and were each evaluated in the same manner as in Example 11 (the results are shown in Table 3 as a list).

TABLE 3

| | |
|---|---|
| | 5-1 |
| 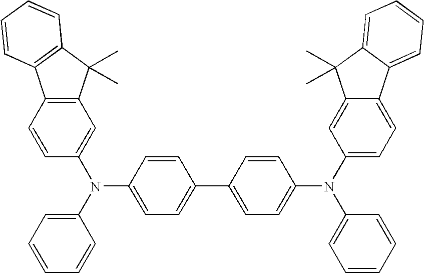 | |

| | | | Initial | | | | |
|---|---|---|---|---|---|---|---|
| | Hole | Hole | | | | | |
| | injection | transport | | | Duration | | |
| | layer | layer | | | (Current | | Luminance |
| | material | material | Applied | | density) | Initial | after |
| | compound | compound | voltage | Luminance | 30.0 | luminance | 100 hours |
| | No. | No. | (V) | (cd/m²) | mA/cm² | (cd/m²) | (cd/m²) |
| Example | 5-1 | C-5 | 5 | 3180 | | 2030 | 1520 |
| 11 | | | | | | | |
| 12 | 5-1 | B-2 | 5 | 3200 | | 2110 | 1590 |
| 13 | 5-1 | D-2 | 5 | 2310 | | 1700 | 1200 |
| 14 | 5-1 | E-5 | 5 | 3140 | | 1980 | 1400 |
| 15 | 5-1 | E-1 | 5 | 3000 | | 1920 | 1360 |

Table 3 below shows the list of the summary of the results of investigation on Examples 11 to 15.

As shown in Table 3, when Compound 5-1 was used as a material for a hole injection layer and a compound described in the present invention was used as a material for a hole transport layer, a good value was obtained for each of the initial luminance of a device and the luminance after 100 hours of the device.

This application claims priority from Japanese Patent Application Nos. 2005-366557 filed Dec. 20, 2005 and 2006-312927 filed Nov. 20, 2006, which are hereby incorporated by reference herein.

1. An amine compound represented by the following general formula (1): 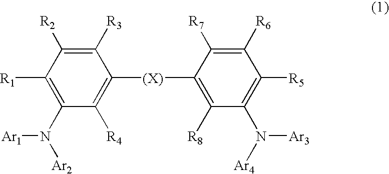
wherein X represents the following general formula (2): 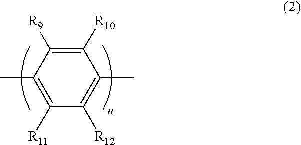
Ar₁, Ar₂, Ar₃ and Ar₄ each independently represent a substituted or unsubstituted aryl group, or a substituted or unsubstituted heterocyclic group, and may be the same or different, and at least one of Ar₁, Ar₂, Ar₃ and Ar₄ represents a substituted or unsubstituted fluorenyl group;
R₁ to R₁₂ each represent a hydrogen atom, a halogen atom, a substituted or unsubstituted alkyl group, a substituted or unsubstituted alkoxy group, a substituted or unsubstituted amino group, a substituted or unsubstituted aryl group, or a substituted or unsubstituted heterocyclic group, and may be the same or different; and
n represents an integer of 0 to 10; and when a plurality of R₉'s, a plurality of R₁₀'s, a plurality of R₁₁'s, or a plurality of R₁₂'s are present, the plurality of R₉'s, the plurality of R₁₀'s, the plurality of R₁₁'s, or the plurality of R₁₂'s may be the same or different, or two of R₉'s to R₁₂'s may form a ring; or X represents a single bond when n represents 0.

2. An amine compound according to claim 1, wherein X in the general formula (1) is represented by the following general formula (3): 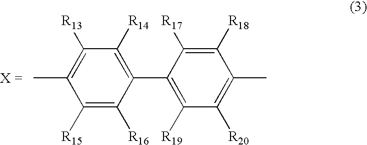
wherein R₁₃ to R₂₀ each represent a hydrogen atom, a halogen atom, a substituted or unsubstituted alkyl group, a substituted or unsubstituted alkoxy group, a substituted or unsubstituted amino group, a substituted or unsubstituted aryl group, or a substituted or unsubstituted heterocyclic group, and may be the same or different.

3. An amine compound according to claim 1, wherein X in the general formula (1) is represented by the following general formula (4): 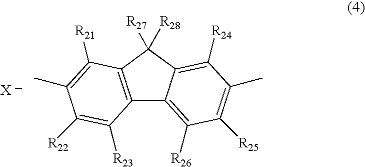
wherein R₂₁ to R₂₈ each represent a hydrogen atom, a halogen atom, a substituted or unsubstituted alkyl group, a substituted or unsubstituted alkoxy group, a substituted or unsubstituted amino group, a substituted or unsubstituted aryl group, or a substituted or unsubstituted heterocyclic group, and may be the same or different.

4. An amine compound according to claim 1, wherein X in the general formula (1) is represented by the following general formula (5): 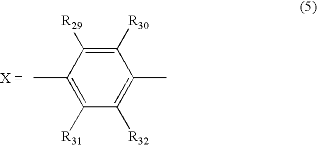
wherein R₂₉ to R₃₂ each represent a hydrogen atom, a halogen atom, a substituted or unsubstituted alkyl group, a substituted or unsubstituted alkoxy group, a substituted or unsubstituted amino group, a substituted or unsubstituted aryl group, or a substituted or unsubstituted heterocyclic group, and may be the same or different.

5. An amine compound according to claim 1, wherein X in the general formula (1) represents a single bond.

6. An organic light-emitting device, comprising an organic compound layer interposed between a pair of electrodes composed of an anode and a cathode, at least one of the anode and the cathode being transparent or semi-transparent, wherein the organic compound layer contains an amine compound according to claim 1.

7. An organic light-emitting device, comprising an organic compound layer as a hole transport layer or a hole injection layer interposed between a pair of electrodes composed of an anode and a cathode, at least one of the anode and the cathode being transparent or semi-transparent, wherein the hole transport layer or the hole injection layer contains an amine compound according to claim 1.

8. An organic light-emitting device, comprising an organic compound layer as a light-emitting layer interposed between a pair of electrodes composed of an anode and a cathode, at least one of the anode and the cathode being transparent or semi-transparent, wherein the light-emitting layer contains an amine compound according to claim 1.

9. An organic blue-light-emitting device, comprising an organic compound layer interposed between a pair of electrodes composed of an anode and a cathode, at least one of the anode and the cathode being transparent or semi-transparent,
wherein a relationship of (an absolute value of an ionization potential of a hole transport material)−(an absolute value of an electron affinity of the hole transport material)≧3.20 eV is established; and
a relationship of (an absolute value of an electron affinity of a host material of use in a light-emitting layer)−(the absolute value of the electron affinity of the hole transport material)≧0.42 eV is established.

10. An organic blue-light-emitting device according to claim 9, wherein a relationship of ((the absolute value of the ionization potential of the hole transport material)−(the absolute value of the electron affinity of the hole transport material))−((an absolute value of an ionization potential of the host material for use in the light-emitting layer)−(the absolute value of the electron affinity of the host material for use in the light-emitting layer))≧0.25 eV is established.

11. An organic blue-light-emitting device according to claim 9, wherein an organic compound for use in a hole transport layer is a triarylamine derivative.

12. An organic light-emitting device, comprising an organic compound layer interposed between a pair of electrodes composed of an anode and a cathode, at least one of the anode and the cathode being transparent or semi-transparent, wherein an organic compound used for a hole transport layer contains at least one kind of an amine compound according to claim 1.
